(19) United States
(12) Patent Application Publication
(10) Pub. No.: US 2019/0364774 A1
(43) Pub. Date: Dec. 5, 2019

(54) MILDEW RESISTANT BASIL PLANTS

(71) Applicant: BAR ILAN UNIVERSITY, Ramat Gan (IL)

(72) Inventors: Yigal COHEN, Kiriat Ono (IL); Yariv BEN NAIM, Yehud (IL)

(21) Appl. No.: 16/477,400

(22) PCT Filed: Jan. 11, 2018
(86) PCT No.: PCT/IL2018/050048

§ 371 Date: Jul. 11, 2019

Related U.S. Application Data
(60) Provisional application No. 62/446,548, filed on Jan. 16, 2017.

(51) Int. Cl. A01H 5/10; A01H 6/50

(52) U.S. Cl. CPC A01H 5/10; A01H 6/506

(57) ABSTRACT

A fertile cultivated basil plant having resistance to basil downy mildew and a method for producing the same.

### FIELD

The present invention relates, inter alia, to Sweet basil (Ocimum basilicum) plants having interspecies introgressed chromosomal region accompanied by sequences from Ocimum ammericanum var amercanum (Canum) into their genome, the sequences conferring resistance to fungal infections, in particular Basil downy mildew (BDM).

### BACKGROUND

Basil downy mildew (BDM) caused by the oomycete foliar pathogen Peronospora belbahrii. is currently the most detrimental disease of sweet basil. Control measures include a few registered fungicides of which the most important one, mefenoxam, has recently become ineffective. Other measures include nocturnal lighting, daytime solar heating and nocturnal fanning.

Basil (Ocimum spp.) includes more than 50 species of herbs and shrubs. To date all commercial sweet basil (O. basilicum) cultivars are highly susceptible to BDM.

Interspecific hybridization and polyploidy occurs commonly within the Ocimum genus, however, interspecies hybrids are sterile due to different ploidy, lack of homology (homeology) between chromosomes or both.

Interspecific crosses would normally produce seeds, which are aborted. Abortion of embryo is derived from interspecific incompatibility caused by genetic distance of parents or different ploidy.

Whereas a gene for BDM resistance exists in the wild inedible Ocimum ammericanum, no resistance gene exists in Ocimum basilicum in nature. Therefore, due to the edible plants being susceptible to BDM, farmers continue to suffer severe losses due to downy mildew.

Certain embodiments of the present disclosure may include some, all, or none of the above advantages. One or more technical advantages may be readily apparent to those skilled in the art from the descriptions and claims included herein. Moreover, while specific advantages have been enumerated above, various embodiments may include all, some or none of the enumerated advantages.

In addition to the exemplary aspects and embodiments described above, further aspects and embodiments will become apparent by study of the following detailed descriptions.

### SUMMARY

Embodiments of the invention disclose incorporation of genetic resistance into crop so as to supply farmers with a relief from diseased crop. Embodiments of the present invention provide sweet basil plants resistant to basil downy mildew.

Advantageously, the sweet basil plants provided herein are fertile and have sequences intogressed into their genome that provide resistance to BDM.

According to some embodiments, the plants are obtained by an interspecific hybridization involving embryo rescue producing fertile and BDM resistant sweet basil plants, as further elaborated herein below.

Advantageously, the aromatic profile of the resistant sweet basil is similar to the aromatic profile of O. basilicum and is devoid of aromatic compounds making wild basil Ocimum ammericanum inedible.

According to some embodiments, there is provided a method for producing a cultivated basil plant having resistance to basil downy mildew (BDM), the method comprising: pollinating a nonresistant basil plant with pollen from a wild resistant basil plant; rescuing fertilized ovules from the nonresistant basil plant; growing the rescued fertilized ovules to F1 plants; backcrossing the F1 plants with the nonresistant basil plant; and selecting for a basil plant having resistance to BDM, wherein the BDM resistant basil plant has introgressed into its genome a sequence conferring resistance to BDM.

According to some embodiments, the nonresistant basil plant comprises sweet basil and the resistant basil plant comprises wild basil. According to some embodiments, the resistant basil plant comprises Ocimum ammericanum. According to some embodiments, the resistant basil plant comprises one of basil accession numbers PI 500945, PI 500950 and PI 652053. According to some embodiments, the sweet basil plant is Ocimum basilicum. The method of claim 1, wherein the non-resistant basil is selected from the group consisting of O. kilimanadascharicum, O. tenuiflorum, O. basilicum O. basilicum var. anisatum, O. basilicum var. thyrsiflorum, O. basilicum var. citrodorum and O. x citrodorum (Syn O. americanum Lemon Types) O. basilicum var. minimum and hybrids thereof.

According to some embodiments, the rescuing comprises: growing a receptacle separated from a sterile basil plant on MS medium at about 25° C. and then at about 18° C.; transferring immature seed to MS medium to develop plantlets; transfer plantlets to rooting medium; and grow plantlets at 27° C. to obtain fertile basil plants.

According to some embodiments, the resistant basil plant is fertile.

According to some embodiments, there is provided a basil plant produced by the method disclosed.

According to some embodiments, there is provided a seed capable of growing into the basil plant disclosed herein.

According to some embodiments, the introgressed sequence conferring the resistance, is from Ocimum ammericanum.

According to some embodiments, the seed is deposited at NCIMB accession number NCIMB-42946.

According to some embodiments, there is provided a plant, explants, scion, cutting, seed, fruit, rootstock, pollen, ovules, and/or plant parts of Ocimum basilicum having in its genome introgressed sequences from Ocimum ammericanum conferring resistance to basil downy mildew (BDM), wherein the plant, explants, scion, cutting, seed, fruit, rootstock, pollen, ovules, and/or plant parts is obtained from a seed as essentially disclosed herein.

According to some embodiments, there is basil downy mildew (BDM) resistant cultivated basil plant and/or seed, comprising a genomic sequence having one or more introgressed nucleic acid sequences conferring resistance or tolerance to BDM relative to a basil plant of the same species lacking the introgressed nucleic acid sequences.

According to some embodiments, the resistant cultivated basil plant is fertile.

According to some embodiments, the cultivated basil plant is edible.

According to some embodiments, the cultivated basil plant and/or seed is selected from the group consisting of O. kihmanadascharicum, O. tenuiflorum, O. basilicum O. basilicum var. anisatum, O. basilicum var. thyrsiflorum, O. basilicum var. citrodorum and O. x citrodorum (Syn O. americanum Lemon Types) O. basilicum var. minimum and hybrids thereof.

According to some embodiments, the cultivated basil plant and/or seed is a Ocimum basilicum plant and/or seed or any hybrid thereof.

According to some embodiments, the one or more introgressed sequences are derived from O. americanum. According to some embodiments, the O. americanum is O. americanum var americanum accession number PI 500945.

According to some embodiments, the one or more introgressed sequences are located at a distance of less than 30 centrimorgan (cM) from a genetic marker having an amino acid sequence selected from SEQ ID NOs: 1-13.

According to some embodiments, the one or more introgressed sequences are located at a distance of less than 30 centrimorgan (cM) from a genetic marker having an amino acid sequence selected from SEQ ID NOs: 1-13. According to some embodiments, the one or more introgressed sequences are located at a distance of less than 20 centrimorgan (cM) from a genetic marker having an amino acid sequence selected from SEQ ID NOs: 1-13.

According to some embodiments, the one or more sequences are homozygously introgressed into the BDM resistant cultivated basil plant and/or seed.

### BRIEF DESCRIPTION OF THE FIGURES

The invention will now be described in relation to certain examples and embodiments with reference to the following illustrative figures so that it may be more fully understood. In the drawings:

FIG. 1A-FIG. 1F: Fluorescent (FIG. 1A, FIG. 1B, FIG. 1D and FIG. 1E) and regular (FIG. 1C, and FIG. 1F) micrographs showing the development Peronospora belbahrii causal agent of BDM in susceptible (FIG. 1A-FIG. 1C) and resistant (FIG. 1D-FIG. 1F) Ocimum species, (bar in FIG. 1A-FIG. 1D is 30 μm, the leaves in FIG. 1A, FIG. 1B, FIG. 1D, and FIG. 1E were stained with basic aniline blue pH8.9 and calcofluor); FIG. 1A shows spore germination and germ-tube penetration into a leaf of the susceptible Ocimum basilicum ‘Sweet basil’ at 1 dpi; FIG. 1B shows haustoria, fluorescing green, in the mesophyll of ‘Sweet basil’ at 7 dpi; FIG. 1C shows sporulation on the lower leaf surface of ‘Sweet basil’ at 7 dpi; FIG. 1D and FIG. 1E show spore germination and germ-tube penetration into a leaf of the resistant Ocimum amercanum var. americanum PI 500945 at 1 dpi. Note the massive callose encasement of the epidermal cell in FIG. 1D and the HR in FIG. 1E; FIG. 1F demonstrates absence of sporulation at 7 dpi on leaves of PI 500945;

FIG. 2 shows a genetic model describing the inheritance of resistance against Peronospora belbahrii in F1 and BCs1 of a cross between the resistant tetraploid accession PI 500945 of Ocimum americanum var. americanum and the susceptible tetraploid Ocimum basilicum ‘Sweet basil’;

FIG. 3 shows a genetic model describing the inheritance of resistance against Peronospora belbahrii in BCs2 (A and B) and BCs1F2 (C) of a cross between the resistant tetraploid accession PI 500945 of Ocimum americanum var. americanum and the susceptible tetraploid Ocimum basilicum ‘Sweet basil’;

FIG. 4 shows a genetic model describing the inheritance of resistance against Peronospora belbahrii in BCs2 F2 and BCs3 of a cross between the resistant tetraploid accession PI 500945 of Ocimum americanum var. americanum and the susceptible tetraploid Ocimum basilicum ‘Sweet basil’;

FIG. 5A-FIG. 5B show response to basil downy mildew (BDM) of susceptible ‘Sweet basil’ plants (FIG. 5A) and resistant BCs4F3 plants (FIG. 5B) under field conditions;

FIG. 6A shows a Mass-Spec Analysis of aromatic compounds of susceptible sweet basil-Ocimum basilicum (upper panel), F1 cross between the resistant tetraploid accession PI 500945 of Ocimum americanum var. americanum and the susceptible tetraploid Ocimum basilicum ‘Sweet basil’ (second panel from above), resistant tetraploid accession PI 500945 of Ocimum americanum (third panel from above), and resistant BCs5F3 plants (lower panel);

FIG. 6B is an exploded view of retention times 7.03-7.52 of FIG. 6A; and

FIG. 6C is an exploded view of retention times 8.08-8.98 of FIG. 6A.

### DETAILED DESCRIPTION OF THE INVENTION

The present invention is directed to fertile sweet basil plants having an altered genotype providing resistance or tolerance to BDM (caused by Peronospora belbahrii), and methods for producing the same. The resistance is facilitated by genetic manipulation resulting in introgression of a gene/nucleic acid sequence into the sweet basil plant genome.

As used herein the term “resistance” or “improved resistance” of a plant to a disease may refer to an indication that the plant is less affected by the disease with respect to yield, survivability and/or other relevant agronomic measures, as compared to a less resistant, more “susceptible” plant. According to some embodiments, resistance is a relative term, indicating that a “resistant” plant survives and/or produces better yields under disease conditions as compared to a different (less resistant) plant. As known in the art, disease “tolerance” is sometimes used interchangeably with disease “resistance.” One of skill in the art will appreciate that plant resistance to disease conditions varies widely, and can represent a spectrum of more-resistant or less-resistant phenotypes. However, by simple observation, one of skill can generally determine the relative resistance or susceptibility of different plants, plant lines or plant families under disease conditions, and furthermore, will also recognize the phenotypic gradations of “resistant”.

As used herein, the term “phenotype” means the detectable characteristics of a cell or organism that can be influenced by gene expression.

As used herein, the term “genotype” refers to the genetic makeup of an individual cell, cell culture, tissue, organism (e.g., a plant), or group of organisms.

As used herein, the term “introgression” when used in reference to a genetic locus, refers to introduction of a nucleic acid sequence into a new genetic background, such as through backcrossing. Introgression of a genetic locus can be achieved through plant breeding methods and/or by molecular genetic methods such as, for a non-limiting example, plant transformation techniques and/or methods that provide for homologous recombination, non-homologous recombination, site-specific recombination, and/or genomic modifications that provide for locus substitution or locus conversion.

As used herein, the term “cross”, “crossing”, “cross pollination” or “cross-breeding” refer to the process by which the pollen of one flower on one plant is applied (artificially or naturally) to the stigma (ovule) of a flower on another plant.

As used herein, the term “locus” (plural: “loci”) refers to any site that has been defined genetically. A locus may be a gene, or part of a gene, or a DNA sequence, and may be occupied by different sequences. A locus may also be defined by a SNP (Single Nucleotide Polymorphism), or by several SNPs. As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

Studies were carried out in order to locate potential sources of resistance to basil downy mildew (BDM) among commercial and wild basil species. Varying levels of resistance/susceptibility to BDM caused by Peronospora belbahrii have been reported for different Ocimum species. wild Ocimum species such as O. americanum, O. kihmanadascharicum, O. gratissimum, O. campechianum, and O. tenuiflorum showed highly resistant; the close relatives of O. basilicum (O. basilicum var. anisatum, O. basilicum var. thyrsiflorum, O. basilicum var. citrodorum, O. x citrodorum and O. basilicum var. minimum) showed moderately resistant to BDM, while all commercial sweet basil (O. basilicum) cultivars showed highly susceptibility to BDM.

As used herein, the terms “variety” and “cultivar” mean a group of similar plants that by their genetic pedigrees and performance can be identified from other varieties within the same species.

As exemplified in the example section below, hybrids showing high resistance to BDM (e.g., F1 hybrids) may be produced by crossing a plant exhibiting resistance to BDM (e.g., plants of: USDA-Plant Introduction number (‘PI’) 500945, PI 500950, PI 500951 and PI 652053) with a plant exhibiting susceptibility to BDM (e.g., sweet basil), notably those hybrids are sterile.

As used herein, the term “hybrid” refers to the offspring or progeny of genetically dissimilar plant parents or stock produced as the result of controlled cross-pollination as opposed to a non-hybrid seed produced as the result of natural pollination.

As used herein, the term “embryo rescue” refers to the development of viable interspecific hybrids from interspecific crosses, which would normally produce seeds which are aborted. Abortion of embryo is derived from interspecific incompatibility caused by genetic distance of parents or different ploidy. Plant embryos may refer to multicellular structures that have the potential to develop into a new plant. In some other cases, the embryo may be a whole ovary plated on media culture. In other cases, zygotic (embryonic) tissue may be extracted from the ovules (coat) and transferred in to a callus tissue culture.

### Methods

According to one aspect, there is provided a method for producing a sweet basil plant having resistance to BDM. In some embodiment, the produced sweet basil plant is fertile. The method includes the steps of interspecies pollination of nonresistant sweet basil plant with pollen from a wild resistant Ocimum plant; rescuing fertilized ovules from the nonresistant basil plant; growing the rescued fertilized ovules to F1 plants; backcrossing the F1 plants with the nonresistant basil plant; and selecting for a basil plant having resistance to downy mildew.

In one embodiment, the invention provides an edible basil plant (Ocimum spp.) having resistance to downy mildew.

As further detailed below a nonresistant basil plant may include sweet basil and a resistant basil plant may be wild basil. In one embodiment, BDM resistant sweet basil were produced by interspecies crosses made between the resistant wild basil O. americanum var americanum PI 500945 and the susceptible Ocimum basilicum sweet basil.

Another embodiment of the invention includes a sweet basil plant comprising a resistance allele from wild basil, which confers resistance to downy mildew.

A further embodiment of the invention includes developing basil plants (Ocimum spp.), by using an embryo rescue system to grow fertile basil plants from sterile basil plants.

In one embodiment the method includes growing a receptacle separated from a sterile basil plant on MS medium at about 25° C. and then at about 18° C.; transferring immature seeds to MS medium to develop plantlets; transferring plantlets to rooting medium; and grow plantlets at about 27° C. to obtain fertile basil plants.

According to some embodiments, there is provided a method for producing a basil plant having resistance to downy mildew, the method comprises: pollinating a nonresistant basil plant with pollen from a wild resistant basil plant; rescuing fertilized ovules from the nonresistant basil plant; growing the rescued fertilized ovules to F1 plants; backcrossing the F1 plants with the nonresistant basil plant; and selecting for a basil plant having resistance to downy mildew.

In some embodiments, the nonresistant basil plant comprises sweet basil and the resistant basil plant comprises wild basil. In some embodiments, the resistant basil plant comprises one of basil accession numbers PI 500945, PI 500950 and PI 652053.

According to some embodiments, there is provided a method of developing basil plants (Ocimum spp.), the method comprises using an embryo rescue system to grow fertile basil plants from sterile basil plants. In some embodiments, the method comprises: growing a receptacle separated from a sterile basil plant on MS medium at about 25° C. and then at about 18° C.; transferring immature seeds to MS medium to develop plantlets; transferring plantlets to rooting medium; and grow plantlets at 27° C. to obtain fertile basil plants.

In some embodiments, the sterile basil plant has resistance to basil downy mildew. In some embodiments, the sterile basil plant is produced by pollinating a nonresistant basil plant with pollen from a wild resistant basil plant.

Embodiments of the disclosure further encompass the plants produced by the methods described herein, seeds capable of growing into the plants, progeny of the plants, propagative material (which may include microspore, pollen, ovary, ovule, embryo, embryo sac, egg cell, cutting, root, root tip, hypocotyl, cotyledon, stem, leaf, flower, anther, seed, meristematic cell, protoplast or cell) derived from the plant, the propagative material capable of growing into a plant according to embodiments of the invention and a tissue culture of the propagative material. Also encompassed are parts of the plants, e.g., a harvested plant or leaf. The part may be in processed form, e.g., a food product or part of a food product or other processed product.

### Compositions

According to some embodiments, there is provided a sweet basil plant or seed capable of growing into a basil plant having a resistance allele from wild basil, which confers resistance to basil downy mildew (BDM). In some embodiments, there is provided a seed capable of growing therefrom a fertile sweet basil plant having resistance to BDM.

According to some embodiments, there is provided a sweet basil plant, comprising: a genomic sequence having one or more introgressed nucleic acid sequences conferring resistance or tolerance to BDM relative to a basil plant of the same species lacking the introgressed nucleic acid sequences. In some embodiments, the sweet basil plant is fertile. In some embodiments, the sweet basil plant is edible. A sample of this BDM resistant Ocimum basilicum (sweet basil) seed has been deposited by the Applicant, Bar Ilan University, Ramat Gan 529002, Israel, pursuant to, and in satisfaction of, the requirements of the Budapest Treaty on the International Recognition of the Deposit of Microorganisms for the Purposes of Patent Procedure (the “Budapest treaty”) with the National Collection of Industrial, Food and Marine Bacteria (NCIMB), (NCIMB Ltd, Ferguson Building, Craibstone Estate, Bucksburn, Aberdeen AB21 9YA, United Kingdom), on Jan. 3, 2017, under accession number NCIMB 42946. The deposited seeds are not from a plant variety. All deposited seeds possess an introgressed fragment and confer the BDM resistant phenotype according to the invention. A plant or seed according to the invention may be a progeny or offspring of a plant grown from the deposited seeds of sweet basil, deposited at the NCIMB under the accession number NCIMB 42946. According to some embodiments, plants grown from the deposited seeds are homozygously resistant to BDM, they thus bear in their genome the introgressed sequences from O. americanum conferring resistance and/or tolerance to BDM. The invention is also directed to resistant plants or seeds as defined above, i.e. containing the introgressed sequences of interest, preferably in homozygous form, obtainable by transferring the introgressed sequences from a resistant sweet basil plant, (representative seeds thereof were deposited under NCIMB accession NCIMB-42946), into another sweet basil genetic background, for example by crossing the resistant plant with a second sweet basil plant parent.

According to some embodiments, the one or more introgressed nucleic acid sequences are obtained from a plant exhibiting resistance to BDM belonging to a species selected from the group consisting of: O. americanum var. americanum and O. americanum var. pilosum. According to some embodiments, the one or more introgressed nucleic acid sequences are obtained from a plant selected from the group consisting of: PI 500945, PI 500950, PI 500951 and PI 652053. According to some embodiments, the one or more introgressed nucleic acid sequences are from O. americanum var americanum PI 500945. According to some embodiments, the one or more introgressed nucleic acid sequences are present homozygously.

As used herein, the terms “homolog” or “homologue” refer to a nucleic acid or peptide sequence which has a common origin and/or functions similarly to a nucleic acid or peptide sequence from another species.

As used herein, the term “homozygote” refers to an individual cell or plant having the same alleles at one or more loci on all homologous chromosomes. As used herein, the term “homozygous” refers to the presence of identical alleles at one or more loci in homologous chromosomal segments.

According to some embodiments, the introgressed sequence conferring the resistance is in linkage disequilibrium with one or more, two or more, three or more, four or more or five or more of the genetic markers selected from (each possibility is a separate embodiment):

SEQ ID NO. 1:
TGCAGGCTACG(C/G)CTTTTGAACTGCTCTGTGAGAAACGAGCATTTCA
TATTACAGATCGGAAGAGCGGTT.
SEQ ID NO. 2:
TGCAGAAG(A/G)TGGAATCTAGGGTTTTGAGCACTTCTTTCGCGAGTTC
GGGGGAAGAAATGACGATTA.
SEQ ID NO. 3:
TGCAGC(A/G)GTGGTGTGAGCAGGTGACGAGAGCGAGC(G/A)TAGCAG
CGGCCGGCGAACCAGAACAGAAATGGA.
SEQ ID NO. 4:
TGCAG(C/A)AGAAGCTTTAGTGCA(C/T)ATAATACTGATGGAGATGGT
TTTTGCCTGACTTCTGTTTGTTGTGCT
SEQ ID NO. 5:
TGCAGGACATT(T/A)TGCAAACTGGAAAAACGATTTTCATCAGCTCAAC
TTTACAGATCGGAAGAGCGGTTC.
SEQ ID NO. 6:
TGCAGAAAACGGAATCTAGGGTTTTCAGCACTTCTTTTGCTAGTTTGGGT
GA(C/A)GAAA(C/T)AAC(G/A)ATTA.
SEQ ID NO. 7:
TGCAGCAAATACGGCTACTGCGG(T/C)AATGGTTCCGTAGGTAAACATA
TTTCCCATTATCTTACAGATCGG.
SEQ ID NO. 8:
TGCAGCATTAGTCCCCGAAGCTCCGGATGTGAA(T/G)ATATGGTTTTTC
TGGAAAGAAAGCGAT(T/C)GAAA(T/A)TC.
SEQ ID NO. 9:
TGCAGTCnTTATATCTAATGATGGGACAAGGACTGAAACCAGTTTCnTCT
GCAAAAGCAGGTAACATCA.
SEQ ID NO. 10:
TGCAGCATGGCACCAAACATGGT(T/C)GCGCATATAATTGCTTGCTTAT
TTGTTATCAGCATTTGCTTCTGT.
SEQ ID NO. 11:
TGCAGCAAGAGGGAGGA(A/G)CAAACGACGCTTACCGATGAGGCTGCCA
TGCAAGCGCTAGCGAGCCA(A/T)GGG.
SEQ ID NO. 12:
TGCAGGTCGAGGAGCTGGTGCTCAAGAGAAAGATCTACAGGGTGGT
(G/A)TACAAGATGGATAGCTCT(C/G)GGA.
SEQ ID NO. 13:
TGCAGTTGAATA(A/T)TCATTTTCTTTCCAAAATTGTTGAG(C/T)AGT
TGGCTGCATAGTCAATTACAGATCGGA.

According to some embodiments, the generic marker may be downstream or upstream to the introgressed sequence conferring the resistance.

The aforementioned genetic markers are found in the deposited seeds NCIMB 42946. A plant according to the invention, or grown from a seed as deposited under accession number NCIMB 42946, is thus particularly valuable in a marker assisted selection for obtaining commercial sweet basil lines and varieties resistant to BDM.

As used herein, the term “linkage disequilibrium” refers to a non-random association of alleles at different loci in a given population and thus describes common inheritance of genomic sequences in a population structure pending on the frequency of recombination.

According to some embodiments, the linkage disequilibrium score may be any positive score, meaning that the association of the genomic markers with the introgressed sequences is not random.

According to some embodiments, the introgressed sequences may have a genetic distance of less than 30 cM, less than 25 cM, less than 20 cM, less than 15 cM, less than 10 cM, or less than 5 cM from the above disclosed genomic markers. Each possibility is a separate embodiment.

As used herein the term “plant” encompasses a whole plant, any part of the plant, a propagation material of the plant or a cell or tissue culture derived from the plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from the plant. Thus, the term “plant” includes whole plants, plant cells, plant protoplast, plant cell or tissue culture from which plants can be regenerated, plant calli, plant clumps and plant cells that are intact in plants or parts of plants, such as seeds, pods, flowers, cotyledons, leaves, stems, buds, roots, root tips and the like.

In some embodiments, the propagation material comprises: a microspore, pollen, ovary, ovule, embryo, embryo sac, egg cell, cutting, root, root tip, hypocotyl, cotyledon, stem, leaf, flower, anther, seed, meristematic cell, protoplast or cell. In some embodiments, the composition includes a tissue culture of the propagation material.

In some embodiments, the part of the basil plant is a harvested plant or leaf, and wherein the part is optionally in processed form. In some embodiments, the part of the basil plant is a food product or part thereof.

The present invention is directed cultivated basil plants and/or seeds (Ocimum Spp.) and their hybrids, plant and seed, resistant to Downy Mildew, Perenospora bellbaharii due to their genome being introgressed with sequences from O. americanum conferring resistance to said disease, when present homozygously or heterozygosly. The introgressed sequences are preferably characterized by defined alleles of SNPs in basil genome. The introgressed sequences can be chosen from those present in the genome of a plant of O. americanum, such as but not limited to O. americanum var americanum accession number PI 500945 PI 500950 or PI 652053. As used herein, the term “cultivated basil plant” may refer to any basil plant used for consumption, tissue culture, hobby, decoration, ornamental use, grafting and the like, such as but not limited to to: O. kilimanadascharicum, O. tenuiflorum, O. basilicum O. basilicum var. anisatum, O. basilicum var. thyrsiflorum, O. basilicum var. citrodorum and O. x citrodorum (Syn O. americanum Lemon Types) O. basilicum var. minimum and hybrids thereof. Each possibility is a separate embodiment. According to some embodiments, the invention is specifically directed to O. basilicum and its hybrids. According to some embodiments, the cultivated basil plant is a sweet basil plant used for consumption.

The invention is also directed to parts of these resistant plants, as well as progeny, to the use of these plants for introgressing the resistance in another genetic background, as well as to different methods for obtaining resistant basils plants or seeds.

A Ocimum basilicum their hybrids, Ocimum Spp. and their hybrids, Ornamental plant, plant and seed having in its genome introgressed sequences from O. americanum conferring resistance to Downy Mildew when present homozygously or heterozygosly, wherein said introgressed sequences are located on homologous or homoelogus chromosomes.

It is expected that during the life of a patent maturing from this application many relevant DNA protectants, sweet basil varieties, sweet basil products and uses will be developed and the scope of the terms provided herein is intended to include all such new technologies a priori.

Advantageously, the aromatic profile of the resistant sweet basil, such as a plant grown from a seed as deposited under accession number NCIMB 42946 is similar to the aromatic profile of O. basilicum and is devoid of aromatic compounds making wild basil Ocimum ammericanum inedible. As a non-limiting example, the resistant sweet basil plant is essentially devoid of alfa Copaene abundant in Ocimum ammericanum. As another non-limiting example, Eugenol is abundant in both O. basilicum and the new resistant sweet basil plant disclosed herein, whereas this compound is absent in Ocimum ammericanum.

As used herein the term “about” refers to ±10%.

The terms “comprises”, “comprising”, “includes”, “including”, “having” and their conjugates mean “including but not limited to”. The term “consisting of means “including and limited to”. The term “consisting essentially of means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Various embodiments and aspects of the present invention as delineated hereinabove and as claimed in the claims section below find experimental support in the following examples. Reference is now made to the following examples, which together with the above descriptions illustrate some embodiments of the invention, however the invention is not limited to the exemplified species.

### Methods and Materials

Germplasm.

A susceptible sweet basil (Ocimum basilicum) Sweet basil and the resistant wild basil (Ocimum americanum var americanum, PI 500945) were used in this example. Plants were grown in multi-cell trays (cell size 2.5 cm) filled with a potting mixture (peat:vermiculite, 1:1, v/v), 1 plant per cell. Before being used, seeds were gently scraped with a sand-paper (P 320) to improve their germination. At the 4-6 leaf stage plants were planted in 1.2×0.5×0.2 m polystyrene containers filled with soil mixture (see above) in a net house covered with 50-mesh white plastic net. During the winter season the net-house was covered with transparent IR (infra-red impermeable) anti-drip polyethylene sheet (Arava type, 100μ width, Polytiv Ltd, Israel).

F1 Cross.

Flowers of adult PI 500945 plants were emasculated and pollinated with pollen taken from adult sweet basil plants. At 5-6 weeks after pollination the F1 seeds were harvested from PI 500945, dried, kept on the bench and used for further studies.

Embryo Rescue.

Adult F1 plants grown in the net-house were completely sterile, failing to produce seeds regardless of the source of pollen used for their pollination. Therefore, the embryo rescue technique developed for Lycopersicon was performed with changes in order to obtain progeny plants from these F1 plants.

F1 plants were pollinated with sweet basil. The flowers, possibly containing fertilized ovules, were excised, disinfected and cultured in 5 cm Petri dishes containing artificial medium. The disinfection process was carried out as follows: flowers were flushed with distilled water for 2 h, placed on sterile filter paper and petals were removed carefully. The receptacle, carrying four immature seeds (nutlets), was separated from the pedicel, placed in 0.3% (v/v) hypochlorite solution for 15 min, washed with sterile water, placed for 5 min in ethanol 70% and rinsed three times with double-distilled sterile water. The cut end of the receptacle was placed on MS medium (Murashige and Skoog, 1962) containing per liter 100 mg/l myo-inositol, 0.4 mg/l thiamine-HCl, 30 g sucrose, and 8 g plant agar pH5.8 (Duchefa Biochemicals, Harlem, The Netherlands). The Petri dished were incubated at 25° C. in the dark for 2 weeks and then at 18° C. (12 h/day, 45 μmol·m⁻²·s⁻¹) for another 2 weeks.

After 4 weeks of incubation some immature seeds were developed. They were transferred onto MS medium amended with 6 mg I-inositol, 20 g sucrose, and 2 mg of 6-benzylaminopurine (BAP) per liter and incubated at 18° C. (12 h/day, 45 μmol·m⁻²·s⁻¹). After 20-30 days small plantlets, most of them having no roots, were developed. Plantlets were transferred to 5.5 cm Petri dishes, 1-2 plantlets per dish, containing rooting medium made of MS salts amended with 1 mg thiamine.HCl, 100 mg I-inositol, 30 g sucrose and 1 mg naphthaleneacetic acid (NAA) per liter. Plates were incubated at 27° C. (16 h/day, 45 μmol·m⁻²·s⁻¹) until plantlets developed lateral roots. The plants were transplanted into Jiffy pots (Jiffy-7®—Peat Pellets and Coco Pellet, www.jiffygroup.com) for acclimation before planting.

Pathogen.

24 isolates of Peronospora belbahrii were collected from the major growing regions in Israel during the years 2012-2015. The isolates collected during 2012 were sensitive to mefenoxam, while those collected during 2013-2015 were mostly resistant to this fungicide. Sweet basil was highly susceptible to all isolates showing disease intensity of 3.2-4 whereas PI 500945 was immune to all the isolates showing no symptoms when inoculated with any isolate. The mefenoxam-resistant isolate K-3 (collected in 2013 at Ein-Tamar, Southern Jordan Valley, Israel) was used in all experiments described below. The isolates were maintained by repeated inoculations of potted sweet basil plants at 20° C.

Inoculation and Disease Assessment in Growth Chambers.

Fresh spores of P. belbahrii were collected from infected plants into cold distilled water, adjusted to 5000 spores/ml and spray-inoculated onto the upper leaf surfaces of the test plants with the aid of a fine glass atomizer. Inoculated plants were placed in a dew chamber at 18° C. in the dark for 15 h to ensure infection and thereafter for 6 days at 25° C. under continuous illumination (60 μmole·m²·s⁻¹) to allow for symptom production. Plants were returned to the dew chamber on the seventh day post inoculation (dpi) to enable sporulation of the pathogen on the inoculated plants. Each plant was visually inspected for disease symptoms and sporulation of the pathogen. Plants showing symptoms and/or sporulation were considered susceptible (S) whereas plants showing neither symptoms nor sporulation were considered resistant (R).

Inoculation and Disease Assessment in the Field.

The inoculated progeny plants (S and R) were transplanted to the net-house together with healthy parental plants. At 7 days after planting, when plants reached the 8-10 leaf stage, they were spray-inoculated with spore suspension (5000 spores/ml) of P. belbahrii with the aid of a hand sprayer. Inoculation took place at 8 pm to ensure high humidity during infection. Starting at one week after inoculation disease records were taken from the inoculated plants. Each plant was visually estimated as described above.

DNA Count in Ocimum Nuclei by Flow Cytometry.

Preparation of nuclei was done according to Arumuganathan and Earle (1991) with modification. The following solutions were used: MgSO4 buffer: 10 mM MgSO4-7.H₂O, 50 mM KCl and 5 mM HEPES (adjust to pH 8.0). Extraction buffer A: MgSO4 buffer amended with 1% (w/v) polyvinylpyrrolidone (PVP-40), 6.5 mM dithiothreitol (DTT), 0.25% (v/v) Triton X-100; stored at 4° C. Extraction buffer B: MgSO4 buffer with amended with 6.5 mM dithiothreitol (DTT). 0.25% (v/v) Triton X-100, 0.2 mg/mL propidium iodide (Acros Organics) and 1.25 μg/mL RNase (DNase-free); prepared on ice just prior to use.

Plants were grown in the greenhouse. Leaves 6 to 8 were cut off from 10 leaf plants, placed in aluminum foil and frozen in liquid nitrogen. The tissue was smashed gently using a mortar and pestle, transferred into a 50 ml tube and kept on ice for ˜10 minutes until thawing of the tissue. Buffer A was added to cover of the tissue and the tube was shaken for 30 min. The extract was transferred to a new tube through a 40 μm mesh sieve strainer (BD Falcon) and centrifuged for 1 min at 11,000 g. The supernatant was discarded and the pellet was suspended in 1.5 ml of Buffer B. The filtration through strainer was repeated and the filtrate was kept at room temp for 30 min for DNA/PI absorption. DNA content of the nuclei was measured by relative fluorescence of samples with a FACScan flow cytometer (Becton Dickinson Immunocytometry Systems-calibure) equipped with an argon-ion laser emitting at 488 nm. Watermelon (C. lanatus var lanatus) 2n and 3n nuclei were used for initial reference calibration. After that initial calibration sweet basil nuclei served as a reference.

Microscopy.

Leaf discs (12 mm diameter) were removed from leaves 6-8 of 10-leaf inoculated plants at 1 dpi. Discs were clarified in boiling ethanol for 10 minutes, placed in basic aniline blue solution (0.05%, pH 8.9) at 4° C. for 24 h, stained with 0.01% calcofluor (Sigma)10 min before being used, and were examined with Olympus A70 epifluorescent microscope for the presence of sporangia and mycelia.

Allelism.

The highly resistant Ocimum americanum Plant Introduction PI 500945 was crossed with the highly resistant Ocimum americanum Plant Introductions PI 500950, PI 500951 and PI 652053.

DNA Analysis

Tissue samples for DNA extraction were taken from 142 BC5 plants for analysis. The DNA samples of all individuals in the BC5 population were sent for enzymatic cutting by the PstI and MseI restriction enzymes in Australia's DArT (Diversity Arrays Technology), which specializes in SNP detection. The raw material received from DArT appeared as an Excel file containing the genotyped, analytical and statistical data. 147 columns represented each of the population plants (142) together with 5 control plants (2 resistant parents, 2 sensitive parents and 1 hybrid (F1) column). Each row represents an SNP from the sequencing, including identifying details of the marker (internal code) and 69 nucleotides representing the sequence from the enzymatic cutting point and SNP's on which polymorphisms are based within the segment. Prior to the introduction of data for statistical analysis and mapping, all markers that did not exhibit polymorphism between the parents, F1 or within the F2 population were filtered. Only 11,229 SNPs of polymorphisms remained. These markers were saved as txt so that we can use them in the next step.

Analysis 1—All the markers and the phenotype of resistance were fed to a statistical analysis to examine the prevalence between markers and the resistance phenotype in K-Means and Hierarchical Clustering analysis. Analysis 2—The txt file was added to the Multi Point software, which was purchased from Prof. Abraham Coroll (University of Haifa), a program designed to build genetic and chromosomal maps, and later analyzing suspicious sites as genes and QTL based on maps using Multi QTL. A detailed and complete working protocol with the software is available on the software homepage: www.multiqtl.com under MultiQTL and Multipoint tabs).

Statistics.

Chi Square tests were performed by using the Excel program. P values of ≥0.05 indicated on acceptance of the suggested inheritance model.

### Example 1

### Microscopy Analysis

Parent's response to inoculation. Potted plants of PI 500945 (O. americanum var americanum) showed no disease symptoms (immunity) upon inoculation in growth chambers with any of the 24 isolates collected during the years 2012-2015. The susceptible sweet basil showed abundant symptoms with massive sporulation when was concurrently inoculated with any of those isolates.

PI 500945 plants growing in net-houses or plastic houses in the field during 4 seasons (during years 2014-2015) developed no disease symptoms all along the growing season (˜120 days) while adjacent sweet basil plants developed abundant symptoms with heavy sporulation in all seasons.

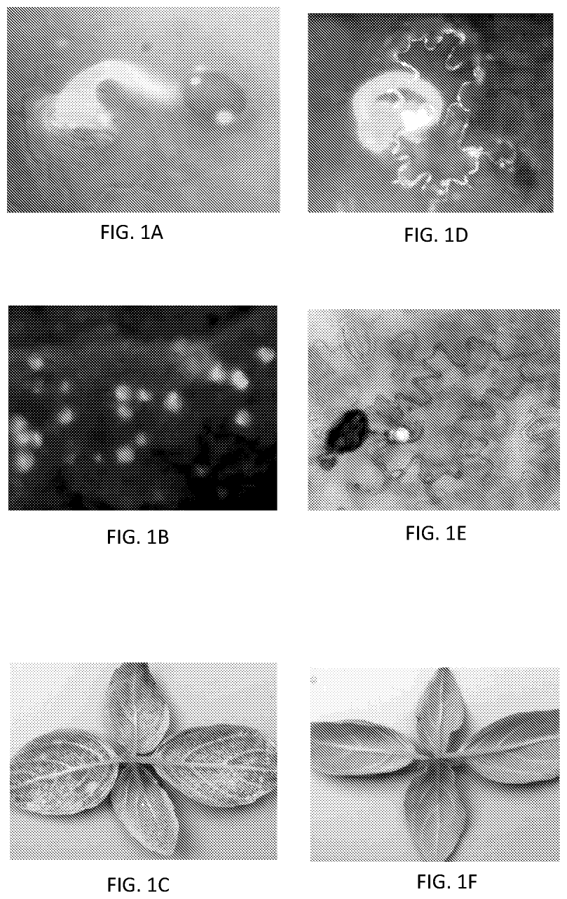

The microscopic responses of the susceptible ‘Sweet basil’ and the resistant PI 500945 to inoculation with isolate Knafo 3 at 1 dpi and 7 dpi are shown in FIG. 1A-FIG. 1F. In ‘Sweet basil’ at 1 dpi, spores of P. belbahrii germinated, produced an appressorium and penetrated into the epidermis via the stomatal opening. No response of the penetrated epidermal cell was observed (FIG. 1A). At 7 dpi, abundant haustoria were seen inside the mesophyll (FIG. 1B) and massive sporulation (˜1×10⁴ spores/mm²) occurred on the lower leaf surface (FIG. 1C). In the resistant PI 500945 at 1 dpi, spores germinated and penetrated equally well into the epidermal cells, but the penetrated cells showed massive accumulation of callose along their cell walls (FIG. 1D). The content of the penetrated cell became dark, producing a hypersensitive response (FIG. 1E). The pathogen stopped developing when the primary vesicle was developed in the epidermal cell (yellow-fluorescing spot in FIG. 1E). A week after inoculation, neither sporophores nor spores were detected in PI 500945 (FIG. 1F). Leaves of F1 plants (PI 500945×‘Sweet basil’) showed similar microscopic responses as the resistant parent PI 500945 (not shown).

TABLE 1
DNA content (mean and standard deviation SD of the mean)
in Ocimum species as determined by Flow cytometry.

| | | pg/ | | Sta- | |
|---|---|---|---|---|---|
| Species | Accession | nucleus | SD | tistic | Ploidy |
| O. americanum | PI 253158 | 2.05 | 0.06 | e | 2n = 2x = 24 |
| var. americanum | | | | | |
| O. basilicum | “Sweet basil’ | 4.66 | 0.05 | d | 2n = 4x = 48 |
| O. basilicum | ‘Aroma 2’ | 4.61 | 0.03 | d | 2n = 4x = 48 |
| O. basilicum | PI 652070 | 4.63 | 0.04 | d | 2n = 4x = 48 |
| O. basilicum | ‘Dark Opal’ | 4.58 | 0.03 | cd | 2n = 4x = 48 |
| O. basilicum | PI 170579 | 4.42 | 0.02 | c | 2n = 4x = 48 |
| var. minimun | | | | | |
| O. americanum | PI 500945 | 4.41 | 0.06 | c | 2n = 4x = 48 |
| var. americanum | | | | | |
| O. basilicum | ‘Mrs. Burns’ | 7.52 | 0.02 | b | 2n = 6x = 72 |
| var. citrodorum | | | | | |
| O. basilicum | ‘Lemon basil’ | 7.5 | 0.02 | b | 2n = 6x = 72 |
| var. citrodorum | | | | | |
| O. basilicum | PI 172997 | 10.41 | 0.01 | a | 2n = 8x = 96 |
| var. anisatum | | | | | |
| O. basilicum | PI 172998 | 10.45 | 0.04 | a | 2n = 8x = 96 |
| var. anisatum | | | | | |

### Example 2

### Inheritance of Resistance

F1 plants of the cross PI 500945 x sweet basil were fully resistant to all 24 isolates used in this study (data not shown). Because such F1 plants were sterile (produced no pollen grains indicating on male sterility and failed to cross with viable pollen of ‘ Sweet basil’ (indicating female sterility), no F2 generation could be produced. To explore the mode of inheritance of resistance, F1 plants were pollinated with pollen of the susceptible sweet basil to obtain BCs1 progeny (first back-cross generation to the susceptible parent). This was achieved by using an embryo rescue technique. A total of 115 BCs1 plants were rescued from about 7,000 flowers.

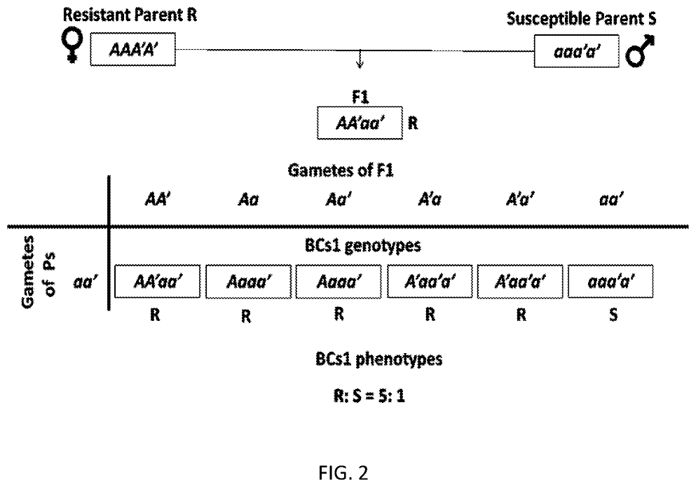

Table 2 shows the response to downy mildew of the susceptible parent ‘Sweet basil’, the resistant parent PI 500945, their F1 plants and their BCs1 progeny. All 46 F1 plants were fully resistant to the disease. BCs1 plants segregated 100 resistant: 15 susceptible. Chi square analysis of four models (one dominant, two dominant, one duplicate dominant and one triplicate dominant genes) suggested an unusual model of 5:1 (R:S). The model indicates that resistance is controlled by a single duplicate dominant gene as shown in FIG. 2. The model suggests that the resistant parent PI 500945 is tetraploid, carrying two copies of a dominant resistance gene A and A′ on two homeologous chromosomes. The corresponding recessive alleles in the susceptible tetraploid parent ‘ Sweet basil’ are a and a′. The F1 AA′aa′ produces 6 types of gametes: AA, Aa, Aa, A′a, A′a′ and aa′. The backcross of F1 to ‘Sweet basil’ produces two phenotypes R and S at a ratio of 5:1 (FIG. 2).

TABLE 2
Four possible models of inheritance of resistance against downy mildew caused by
Peronospora belbahrii in BCs1 obtained from a cross between the resistant wild basil
Ocimum americanum var. americanum PI 500945 and the susceptible Ocimum
basilicum ‘Sweet basil’

| | | Observed | | Expected | | | | | |
|---|---|---|---|---|---|---|---|---|---|
| | No. | ratio | | ratio | | Tested | | | |
| Pedigree | plants | R | S | R | S | ratio | Gene(s) | P | x² |
| PI 500945 | 20 | 20 | — | — | — | — | — | — | — |
| (R-Parent) | | | | | | | | | |
| “Sweet | 20 | — | 20 | — | — | — | — | — | — |
| basil’ | | | | | | | | | |
| (S-Parent) | | | | | | | | | |
| R × S, F1 | 46 | 46 | | — | — | — | — | — | — |
| BCs1 | 115 | 100 | 15 | 57.5 | 57.5 | 1:1 | 1 dominant | 2.258E−15 | 8.008E−30 |
| BCs1 | 115 | 100 | 15 | 86 | 29 | 3:1 | 2 dominant | 0.0026 | 1.099E−05 |
| BCs1 | 115 | 100 | 15 | 96 | 19 | 5:1 | 1 duplicate | 0.3162* | 0.165 |
| | | | | | | | dominant | | |
| BCs1 | 115 | 100 | 15 | 109 | 6 | 19:1 | 1 triplicate | 0.00016 | 4.054E−08 |
| | | | | | | | dominant | | |

*Accepted (P > 0.05)

Results presented in Table 2 show two modes of segregation, 1:1 and 5:1 R:S. The ratio between the two modes of inheritance was 4:1 [(1:1):(5:1)]. Plants were transplanted to a net house at 8 dpi and disease records were taken again at 1, 2 and 3 months after transplanting. The response to disease that was recorded in growth chambers (R or S) was maintained in the field all along the season.

Because BCs1 plants were sterile, they were pollinated (backcrossed) with ‘Sweet basil’ Twenty-two BCs2 progenies were obtained (second backcross generation to the susceptible parent). BCs2 plants at the 4-6 leaf stage (8-46 plants per progeny) were inoculated with P. belbahrii in growth chambers and their response to BDM was evaluated at 7 dpi. The results are presented in Table 3. Progenies showed one of two different modes of R:S segregation, either 5:1 or 1:1. The ratio between the two modes was 1:4 [(5:1):(1:1)].

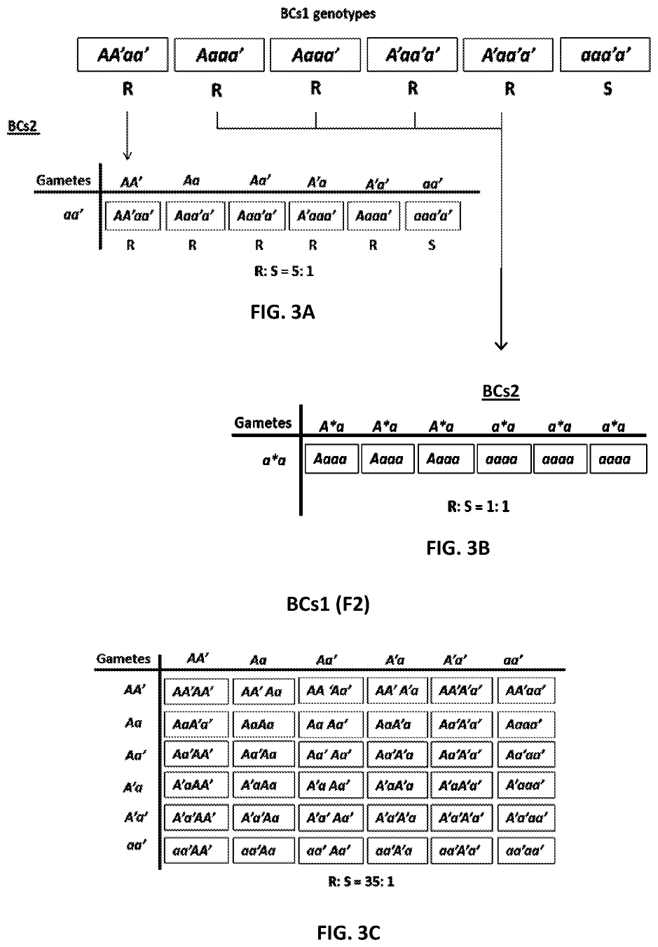

A genetic model supporting the BCs2 data presented in Table 3 is illustrated FIG. 3. It shows that the backcross AA′aa′×aa′aa′ yields a 5:1 R:S segregating progeny (FIG. 3A), while the other four backcrosses Aaaa′×aa′aa′, Aaaa′×aa′aa′, A′aa′a′×aa′aa′ and A′aa′a′×aa′aa′ yield 1:1 R:S segregating progenies (FIG. 3B). The backcross of the susceptible BCs1 aa′a′a to ‘Sweet basil’ aa′a′a yields a susceptible progeny.

A single plant, BCs1-1, was fertile, enabling self-pollination AA′aa′×AA′aa′. Its progeny plants segregated 35:1, R:S (bottom of Table 3), confirming that resistance is controlled by a duplicate dominant gene (Table 3; FIG. 3C).

TABLE 3
Inheritance of resistance against downy mildew Peronospora belbahrii
in 22 BCs2 progenies derived from a cross between the resistant
accession PI 500945 of Ocimum americanum var. americanum and the
susceptible Ocimum basilicum ‘Sweet basil’

| | | Observed | | Expected | | | | | |
|---|---|---|---|---|---|---|---|---|---|
| | No. | ratio | | ratio | | Tested | | | |
| Pedigree | plants | R | S | R | S | ratio | Genes | P | x² |
| PI 500945 | 17 | 17 | 0 | — | — | — | — | — | — |
| (R-Parent) | | | | | | | | | |
| “Sweet basil’ | 48 | 0 | 48 | — | — | — | — | — | — |
| (S-Parent) | | | | | | | | | |
| R × S, F1 | 25 | 25 | 0 | — | — | — | — | — | — |
| BCs2(1) | 46 | 34 | 12 | 38 | 8 | 5:1 | 1 | 0.120 | 0.023 |
| | | | | | | | duplicate | | |
| | | | | | | | dominant | | |
| BCs2(2) | 23 | 11 | 12 | 11.5 | 11.5 | 1:1 | 1 | 0.835 | 1.926 |
| | | | | | | | dominant | | |
| BCs2(3) | 21 | 9 | 12 | 10.5 | 10.5 | 1:1 | 1 | 0.513 | 0.482 |
| | | | | | | | dominant | | |
| BCs2(4) | 28 | 16 | 12 | 14 | 14 | 1:1 | 1 | 0.450 | 0.357 |
| | | | | | | | dominant | | |
| BCs2(6) | 31 | 16 | 15 | 15.5 | 15.5 | 1:1 | 1 | 0.857 | 2.150 |
| | | | | | | | dominant | | |
| BCs2(7) | 24 | 13 | 11 | 12 | 12 | 1:1 | 1 | 0.683 | 1.002 |
| | | | | | | | dominant | | |
| BCs2(9) | 8 | 9 | 1 | 7.5 | 2.5 | 5:1 | 1 | 0.273 | 0.122 |
| | | | | | | | duplicate | | |
| | | | | | | | dominant | | |
| BCs2(10) | 29 | 11 | 17 | 14.5 | 14.5 | 1:1 | 1 | 0.259 | 0.109 |
| | | | | | | | dominant | | |
| BCs2(12) | 27 | 15 | 12 | 13.5 | 13.5 | 1:1 | 1 | 0.564 | 0.606 |
| | | | | | | | dominant | | |
| BCs2(13) | 17 | 13 | 4 | 14 | 3 | 5:1 | 1 | 0.525 | 0.509 |
| | | | | | | | duplicate | | |
| | | | | | | | dominant | | |
| BCs2(15) | 43 | 13 | 30 | 21.5 | 21.5 | 1:1 | 1 | 0.010a | 0.00014 |
| | | | | | | | dominant | | |
| BCs2(16) | 36 | 21 | 15 | 18 | 18 | 1:1 | 1 | 0.317 | 0.167 |
| | | | | | | | dominant | | |
| BCs2(17) | 19 | 12 | 7 | 9.5 | 9.5 | 1:1 | 1 | 0.251 | 0.103 |
| | | | | | | | dominant | | |
| BCs2(19) | 21 | 16 | 5 | 17 | 4 | 5:1 | 1 | 0.578 | 0.646 |
| | | | | | | | duplicate | | |
| | | | | | | | dominant | | |
| BCs2(20) | 34 | 21 | 13 | 17 | 17 | 1:1 | 1 | 0.170 | 0.046 |
| | | | | | | | dominant | | |
| BCs2(21) | 17 | 12 | 5 | 14 | 3 | 5:1 | 1 | 0.203 | 0.066 |
| | | | | | | | duplicate | | |
| | | | | | | | dominant | | |
| BCs2(22) | 22 | 13 | 9 | 11 | 11 | 1:1 | 1 | 0.394 | 0.266 |
| | | | | | | | dominant | | |
| BCs2(24) | 15 | 9 | 6 | 7.5 | 7.5 | 1:1 | 1 | 0.439 | 0.337 |
| | | | | | | | dominant | | |
| BCs2(26) | 43 | 23 | 20 | 21.5 | 21.5 | 1:1 | 1 | 0.647 | 0.864 |
| | | | | | | | dominant | | |
| BCs2(29) | 28 | 12 | 16 | 14 | 14 | 1:1 | 1 | 0.450 | 0.357 |
| | | | | | | | dominant | | |
| BCs2(30) | 26 | 15 | 11 | 13 | 13 | 1:1 | 1 | 0.433 | 0.327 |
| | | | | | | | dominant | | |
| BCs2(31) | 37 | 20 | 17 | 18.5 | 18.5 | 1:1 | 1 | 0.622 | 0.777 |
| | | | | | | | dominant | | |
| (BCs1-1) × | 29 | 27 | 2 | 28 | 1 | 35:1 | 1 | 0.309 | 0.158 |
| (BCs1-1)b | | | | | | | duplicate | | |
| | | | | | | | dominant | | |

aA single pedigree for which none of the models was accepted (P < 0.05).
bSelf-pollinated pedigree of a fertile BCs1-1.

All inoculated progenies plants were transplanted to a net-house at 8 dpi and disease records were taken at 1, 2 and 3 months after transplanting. The response to disease recorded in the growth chambers (Resistant—R or Susceptible—S) was maintained in the field all along the season.

BCs2 progenies plants segregated 19 fertile: 16 sterile. The 19 fertile plants were both self-pollinated and backcrossed to ‘Sweet basil’. The 16 sterile plants were discarded.

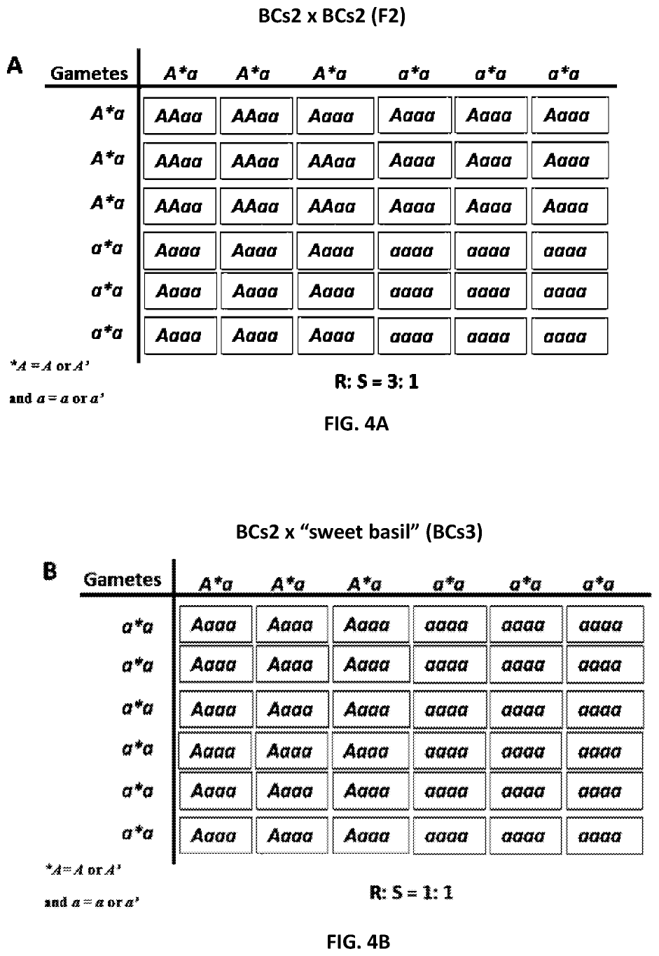

The data presented in Table 4 and FIG. 4A show that 18 out of 19 self-pollinated BCs2 (A*a*aa×A*a*aa) progenies segregated R:S at a ratio of 3:1, suggesting that a single dominant gene controls resistance in BCs2 plants. (Note that A*=A or A′ and a*=a or a′)

Sixteen BCs2 plants (of the 19 fertile plants) were also backcrossed to ‘Sweet basil’ (A*a*aa×a*a*aa) to obtain BCs3 progenies.

TABLE 4
Inheritance of resistance against downy mildew Peronospora
belbahrii in 19 BCs2 × BCs2 (self) progenies obtained from
7 BCs2 progenies of the cross between the resistant accession
Ocimum americanum var. americanum PI 500945 and the
susceptible Ocimum basilicum ‘Sweet basil’

| | | | Observed | | Expected | | |
|---|---|---|---|---|---|---|---|
| | BCs2 × BCs2 | No. | ratio | | ratio | | P |
| BCs1 | (self) | plants | R | S | R | S | for 3:1 |
| 1 | 2 | 10 | 8 | 2 | 7.5 | 2.5 | 0.715 |
| | 3 | 30 | 19 | 11 | 22.5 | 7.5 | 0.140 |
| | 5 | 22 | 15 | 7 | 16.5 | 5.5 | 0.460 |
| | 15 | 10 | 10 | 0 | 7.5 | 2.5 | 0.068 |
| | 16 | 12 | 9 | 3 | 9 | 3 | 1 |
| | 20 | 13 | 10 | 3 | 9.75 | 3.25 | 0.872 |
| | 22 | 40 | 34 | 6 | 30 | 10 | 0.145 |
| | 27 | 57 | 43 | 14 | 42.75 | 14.25 | 0.940 |
| | 29 | 83 | 59 | 24 | 62.25 | 20.75 | 0.410 |
| | 30 | 27 | 17 | 10 | 20.25 | 6.75 | 0.149 |
| 2 | 8 | 17 | 11 | 6 | 12.75 | 4.25 | 0.327 |
| 3 | 1 | 49 | 47 | 2 | 37 | 12 | 0.00071* |
| | 5 | 14 | 12 | 2 | 10.5 | 3.5 | 0.355 |
| | 10 | 2 | 2 | 0 | 1.5 | 0.5 | 0.414 |
| 4 | 3 | 21 | 15 | 6 | 15.75 | 5.25 | 0.705 |
| 6 | 3 | 15 | 11 | 4 | 11.25 | 3.75 | 0.882 |
| 17 | 1 | 2 | 1 | 1 | 1.5 | 0.5 | 0.414 |
| 26 | 3 | 6 | 4 | 2 | 4.5 | 1.5 | 0.640 |
| | 1 | 7 | 4 | 3 | 5.25 | 1.75 | 0.276 |

*Unaccepted (P < 0.05) for 3:1 but accepted for 35:1 (P = 0.57)

The data presented in Table 5 and FIG. 4B show that all BCs3 progenies segregated R:S at a ratio of 1:1, reaffirming that a single dominant gene controls resistance in BCs2 plants. Four BCs3 progenies segregated R:S at a ratio of 5:1, probably because one homeologous chromosome (carrying resistance) has not yet been replaced by a ‘susceptible’ one.

TABLE 5
Inheritance of resistance against downy mildew Peronospora belbahrii
in 35 BCs3 progenies derived from 11 BCs2 progenies of the cross between
the resistant accession Ocimum americanum var. americanum PI 500945
and the susceptible Ocimum basilicum ‘Sweet basil’

| | | | Observed | | Expected | | | |
|---|---|---|---|---|---|---|---|---|
| | BCs2 × ‘Sweet basil’ | No. | ratio | | ratio | | P | P |
| BCs1 | (BCs3) | plants | R | S | R | S | for 1:1 | for 5:1 |
| 1 | 2 | 22 | 10 | 12 | 11 | 11 | 0.670 | |
| | 5 | 13 | 7 | 6 | 6.5 | 6.5 | 0.782 | |
| | 6 | 30 | 24 | 6 | 15 | 15 | 0.001* | 0.624** |
| | 12 | 8 | 3 | 5 | 4 | 4 | 0.480 | |
| | 14 | 39 | 21 | 18 | 19.5 | 19.5 | 0.631 | |
| | 15 | 15 | 11 | 4 | 7.5 | 7.5 | 0.071 | |
| | 16 | 29 | 15 | 14 | 14.5 | 14.5 | 0.853 | |
| | 19 | 18 | 4 | 14 | 9 | 9 | 0.019*** | 00000*** |
| | 21 | 11 | 11 | 0 | 5.5 | 5.5 | 0.001* | 0.117** |
| | 22 | 37 | 21 | 16 | 18.5 | 18.5 | 0.411 | |
| | 23 | 11 | 7 | 4 | 5.5 | 5.5 | 0.366 | |
| | 24 | 30 | 13 | 17 | 15 | 15 | 0.465 | |
| | 27 | 13 | 10 | 3 | 6.5 | 6.5 | 0.052 | 0.53** |
| | 29 | 62 | 28 | 34 | 31 | 31 | 0.446 | |
| | 30 | 33 | 13 | 20 | 16.5 | 16.5 | 0.22 | |
| 2 | 1 | 25 | 13 | 12 | 12.5 | 12.5 | 0.841 | |
| | 4 | 18 | 8 | 10 | 9 | 9 | 0.637 | |
| | 8 | 30 | 14 | 16 | 15 | 15 | 0.715 | |
| | 11 | 20 | 10 | 10 | 10 | 10 | 1.000 | |
| 3 | 1 | 22 | 16 | 6 | 11 | 11 | 0.033* | 0.268** |
| | 5 | 33 | 14 | 19 | 16.5 | 16.5 | 0.384 | |
| | 8 | 20 | 13 | 7 | 10 | 10 | 0.180 | |
| 4 | 1 | 4 | 1 | 3 | 2 | 2 | 0.317 | |
| 6 | 5 | 9 | 2 | 7 | 4.5 | 4.5 | 0.096 | |
| | 11 | 6 | 4 | 2 | 3 | 3 | 0.414 | |
| | 12 | 22 | 12 | 10 | 11 | 11 | 0.670 | |
| | 18 | 15 | 8 | 7 | 7.5 | 7.5 | 0.796 | |
| 9 | 2 | 13 | 4 | 9 | 6.5 | 6.5 | 0.166 | |
| 10 | 4 | 6 | 3 | 3 | 3 | 3 | 1.000 | |
| 15 | 5 | 11 | 4 | 7 | 5.5 | 5.5 | 0.366 | |
| 17 | 3 | 25 | 10 | 15 | 12.5 | 12.5 | 0.317 | |
| 21 | 3 | 6 | 2 | 4 | 3 | 3 | 0.414 | |
| 26 | 1 | 7 | 4 | 3 | 3.5 | 3.5 | 0.705 | |
| | 3 | 34 | 22 | 12 | 17 | 17 | 0.086 | |
| | 16 | 8 | 2 | 6 | 4 | 4 | 0.157 | |

*The model 1:1 unaccepted (P < 0.05).
**The model 5:1 accepted (P < 0.05).
***Both models unaccepted

Four single plants of four BCs3 families 1/27/1, 1/27/4, 1/27/9 and 4/1/5 were self-pollinated or backcrossed to ‘Sweet basil’ and large offspring populations tested for response to BDM. BCs3×BCs3 1/27/1 and 1/27/4 produced 621 resistant and 26 susceptible plants (35:1) whereas BCs3×‘Sweet basil’ produced 327 resistant and 74 susceptible plants, confirming that each carries one duplicate dominant resistance gene (Table 6). BCs3×BCs3 1/27/9 and 4/1/5 produced 164 resistant and 62 susceptible plants (3:1) whereas BCs3×‘Sweet basil’ produced 131 resistant and 114 susceptible plants (1:1), confirming that each carry one dominant resistance gene (Table 6).

TABLE 6
Inheritance of resistance against downy mildew Peronospora belbahrii in large
populations of BCs3 × BCs3 (self) and BCs4 families derived from the cross
between the resistant accession Ocimum americanum var. americanum PI 500945
and the susceptible Ocimum basilicum ‘Sweet basil’

| | | Observed | | Expected | | | | | |
|---|---|---|---|---|---|---|---|---|---|
| | No. | ratio | | ratio | | Tested | | | |
| Pedigree | plants | R | S | R | S | ratio | Gene(s) | P | x² |
| PI 500945 | 10 | 10 | — | — | — | — | — | — | — |
| (R-Parent) | | | | | | | | | |
| ‘Sweet basil’ | 15 | — | 15 | — | — | — | — | — | — |
| (S-Parent) | | | | | | | | | |
| R × S, F1 | 10 | 10 | | — | — | — | — | — | — |
| BCs3 | 311 | 297 | 14 | 302 | 9 | 35:1 | 1 duplicate | 0.091 | 0.013 |
| (1/27/1) Self | | | | | | | dominant | | |
| BCs4 (1/27/1 × | 80 | 71 | 9 | 67 | 13 | 5:1 | 1 duplicate | 0.23 | 0.082 |
| S.b) | | | | | | | dominant | | |
| BCs3 | 336 | 324 | 12 | 327 | 9 | 35:1 | 1 duplicate | 0.38 | 0.24 |
| (1/27/4) Self | | | | | | | dominant | | |
| BCs4 (1/27/4 × | 321 | 256 | 65 | 267 | 54 | 5:1 | 1 duplicate | 0.1 | 0.016 |
| S.b) | | | | | | | dominant | | |
| BCs3 Self - | 647 | 621 | 26 | 629 | 18 | 35:1 | 1 duplicate | 0.056 | 0.005 |
| Total | | | | | | | dominant | | |
| BCs4 - Total | 401 | 327 | 74 | 334 | 67 | 5:1 | 1 duplicate | 0.35 | 0.2 |
| | | | | | | | dominant | | |
| BCs3 | 122 | 89 | 33 | 91.5 | 30.5 | 3:1 | 1 dominant | 0.32 | 0.16 |
| (1/27/9) Self | | | | | | | | | |
| BCs4 (1/27/9 × | 109 | 60 | 49 | 54.5 | 54.5 | 1:1 | 1 dominant | 0.29 | 0.14 |
| S.b) | | | | | | | | | |
| BCs3 (4/1/5) | 104 | 75 | 29 | 78 | 26 | 3:1 | 1 dominant | 0.5 | 0.45 |
| Self | | | | | | | | | |
| BCs4 (4/1/5 × | 136 | 71 | 65 | 68 | 68 | 1:1 | 1 dominant | 0.6 | 0.73 |
| S.b) | | | | | | | | | |
| BCs3 Self - | 226 | 164 | 62 | 167.5 | 56.5 | 3:1 | 1 dominant | 0.43 | 0.33 |
| Total | | | | | | | | | |
| BCs4 - Total | 245 | 131 | 114 | 122.5 | 122.5 | 1:1 | 1 dominant | 0.28 | 0.13 |

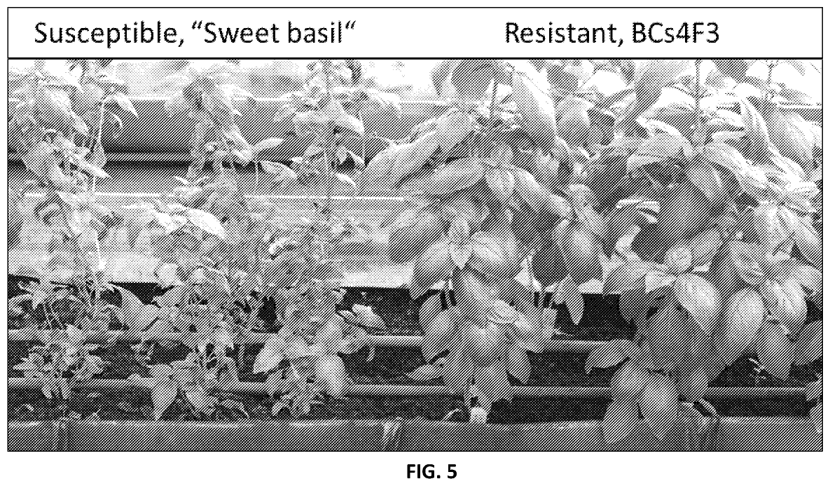

Compared with susceptibility of ‘Sweet basil’ (FIG. 5A), BCs4 resistant lines which were subjected to F2-F3 progeny test provided homozygous BDM-resistant lines (FIG. 5B) exhibiting high yield and good aroma. The BCs4 resistant lines tested were derived from a cross between the resistant tetraploid accession PI 500945 of Ocimum americanum var. americanum and the susceptible tetraploid Ocimum basilicum ‘Sweet basil’.

The sterility barrier of F1 was overcome by using embryo rescue technology. Surprisingly, in-spite of the very low rate of success the inventors were able to produce 115 BCs1 plants [(PI 500945 x sweet basil) x sweet basil] of which 100 plants were fully resistant (immune) and 15 plants susceptible to the disease. This unusual 5:1 segregation ratio was analyzed for fit to a 2n, 4n or 6n model. The 4n model was the only model accepted suggesting that resistance in BCs1 is controlled by a duplicate single dominant gene Pb1 carried by two, probably identical, chromosomes.

Offspring plants of the second back-cross (BCs2) to sweet basil {[(P1500945 x sweet basil) x sweet basil] x sweet basil} showed two modes of segregation, 5:1 R:S and 1:1 R:S, with a respective ratio of 4:1. The flow chart presented in FIG. 3 shows the gametes and genotypes of theses progenies, based on the assumption that both parents are tetraploid.

BCs2 plants showed restored fertility, therefore self-pollinated to obtain BCs2-F2 progenies. All progenies, except one, segregated 3:1 R:S suggesting a single dominant gene controlling resistance in BCs2. The restoration of fertility and the change in the mode of inheritance indicated that one of the duplicate chromosomes derived from the resistant parent (carrying Pb1) has dissipated after 3 crosses to Sweet basil.

To further increase quality of the resistant basil, BCs2 plants were again back-crossed, for the third time, to Sweet basil.

All F1 and F2 progeny plants of the cross PI 500945×PI 500950, and all progeny plants of the cross PI 500945×PI 652053 were highly resistant to BDM as were the parental lines. F1 pedigree plants of the cross PI 500945×PI 500951 were resistant to BDM, but the F2 pedigree plants segregated into resistant, moderately resistant and susceptible plants. These data suggest that Plant Introductions PI 500945, PI 500950, PI 652053 carry the same gene for resistance against BDM.

### Example 3

### DNA Analysis

Following analysis of the 11,229 markers of SNP's polymorphisms, 13 markers (SEQ IDs 1-13) were found to be in linkage disequilibrium with a BDM resistance phenotype based on K-Mean and Hierarchical Clustering analysis and on MultiQTL and Multipoint mapping analysis.

Approximately half of the markers were identified as being downstream the introgressed sequence conferring resistance, the other half, upstream. The introgressed sequences conferring resistance had a genetic distance of less than 30 cM from all the above disclosed genomic markers. Certain markers had a genomic distance of less than 10 cM (SEQ ID NO: 4, SEQ ID NO: 5, SEQ ID NO: 6, SEQ ID NO: 7, SEQ ID NO: 8, SEQ ID NO: 9 and SEQ ID NO: 10) to the genomic locus conferring resistance, some of these a genomic distance of less than 5 cM.

### Example 4—Aromatic Profile

### Method and Parameters

Perkin Elmer CLARUS 680 GS (Gas Chromatography) and mass spectrometer Clarus SQ 8C were utilized. The initial temperature was 35° C., isotherm of 3 min. The temperature was then elevated at a rate of 30° C./min until 250° C. The range of masses was 25-400 Da, EI+. The aroma was measured by 2 min of SPME (Solid Phase Micro Extraction) and two min in the GC injector (250° C.) before GC analysis.

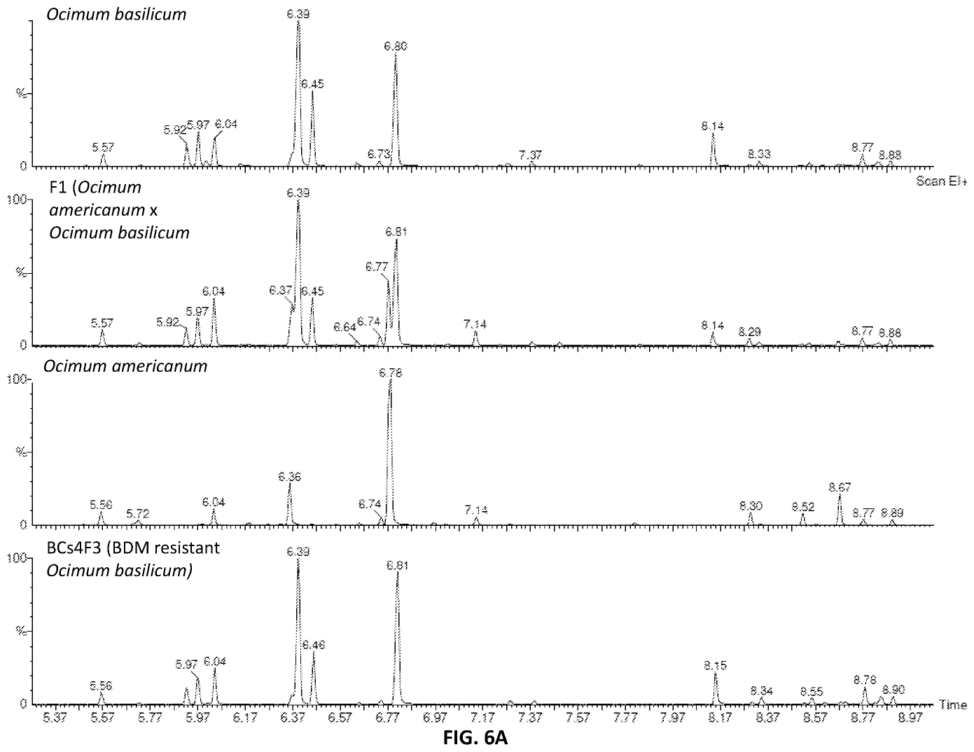

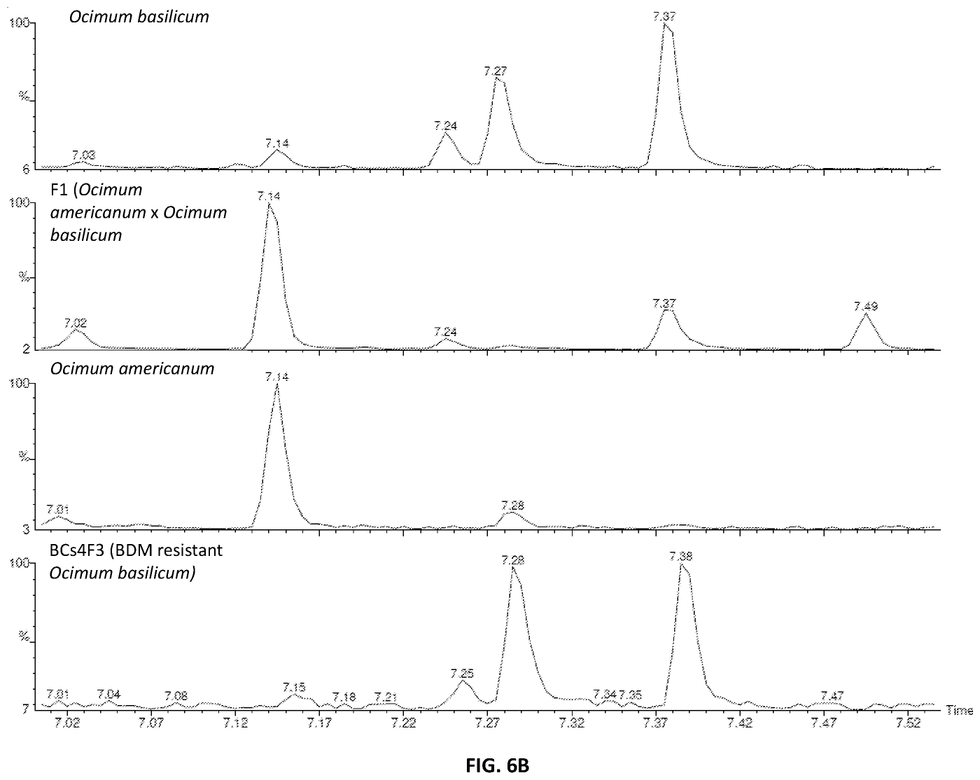

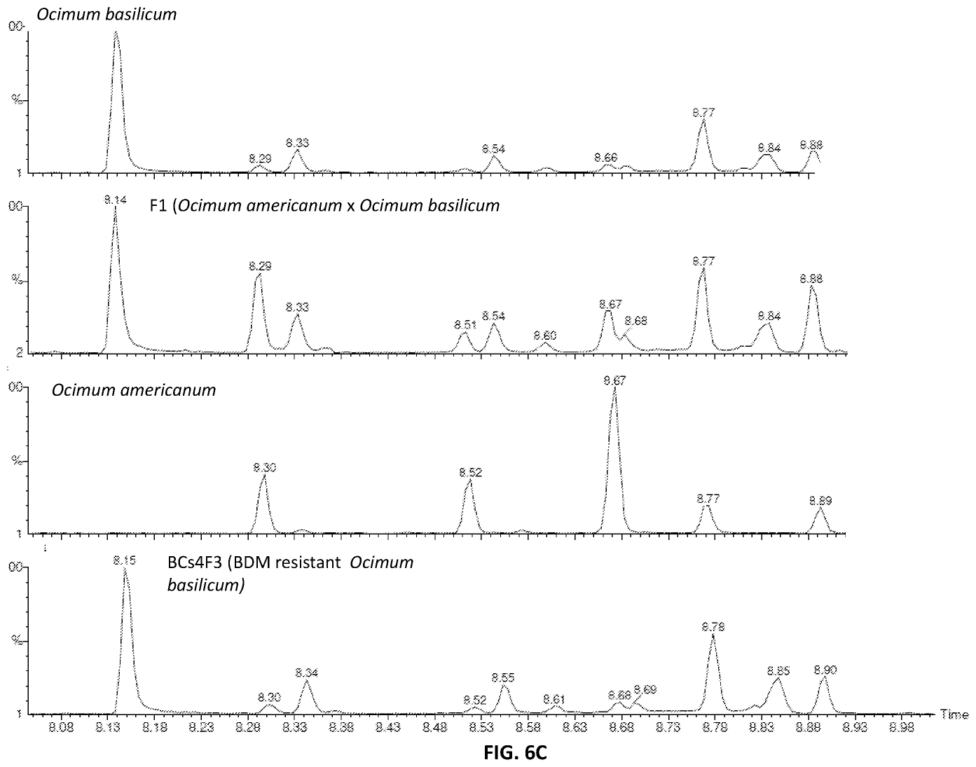

As seen from FIGS. 6A-6C and from table 7 below, the aromatic profile of the resistant sweet basil is similar to the aromatic profile of O. basilicum and is devoid of aromatic compounds making wild basil Ocimum ammericanum inedible.

TABLE 7
Retention time of Basil aromatic compaounds

| Compound | Retention time |
|---|---|
| Sabinen | 5.92 |
| ß-Pinene | 5.97 |
| ß-Geraniolene | 6.04 |
| Octan-3-One | 6 |
| Eucalyptol | 6.39 |
| (+) D-Limonene | 6.36 |
| ß-0 cimene | 6.45 |
| Terpinene 4 acetate | 6.63 |
| à-Terpinolene | 6.73 |
| fenchon | 6.77 |
| fenchon | 6.78 |
| Linalool | 6..8 |
| Camphor | 7.14 |
| Terpineol | 7.24 |
| Camphol | 7.27 |
| terpineol | 7.37 |
| Bornyl Acetate | 7.87 |
| Eugenol | 8.14 |
| alfa Copaene | 8.3 |
| à-Guaiene | 8.54 |
| ß-Copanene | 8.77 |
| à-Muurolene | 8.88 |

For example, the herein disclosed fertile and BDM resistant sweet basil (BCs4) plant is essentially devoid of alfa Copaene abundant in Ocimum ammericanum. Oppositely, Eugenol and terpineol are abundant in both O. basilicum and the new BDM resistant sweet basil plant disclosed herein, whereas these compounds are essentially absent in Ocimum ammericanum.

Advantageously, the BDM resistant sweet basil plant remains edible despite having introgressed into its genome sequences from the inedible Ocimum ammericanum.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

Ben-Naim, Y., Falach, L., and Cohen, Y. 2015a. Resistance to Peronospora belbahrii in wild Ocimum species and its introgression into sweet basil. Phytoparasitica 43:371.
Ben-Naim, Y., Falach, L., and Cohen, Y. 2015b. Resistance against basil downy mildew in Ocimum species. Phytopathology 105:778-785.
Cohen, Y., Ben-Naim, Y., Falach, L., and Rubin, A. V. 2017. Epidemiology of basil downy mildew. Phytopathology 107:1149-1160.
Farahani-Kofoet, R. D., Römer, P., and Grosch, R. 2014. Selecting basil genotypes with resistance against downy mildew. Scientia Horticulturae 179:248-255.
Wyenandt, C. A., Simon, J. E., McGrath, M. T., and Ward, D. L. 2010. Susceptibility of Basil cultivars and breeding lines to downy mildew (Peronospora belbahrii). HortScience 45:1416-1419.
Koroch, A. R., Wang, W., Michael, T. P., Dudai, N., Simon, J. E., and Belanger, F. C. 2010. Estimation of nuclear DNA content of cultivated Ocimum species by using flow cytometry. Isr. J. Plant Sci. 58:183-189.
Rewers, M., and Jedrzejczyk, I. 2016. Genetic characterization of Ocimum genus using flow cytometry and inter-simple sequence repeat markers. Industrial Crops Products 91:142-151.

1.-24. (canceled)

25. A method for producing a cultivated basil plant having resistance to basil downy mildew (BDM), the method comprising:
pollinating a nonresistant basil plant with pollen from a wild resistant basil plant;
rescuing fertilized ovules from the nonresistant basil plant;
growing the rescued fertilized ovules to F1 plants;
backcrossing the F1 plants with the nonresistant basil plant; and selecting for a basil plant having resistance to BDM, wherein the BDM resistant basil plant has introgressed into its genome a sequence conferring resistance to BDM.

26. The method of claim 25, wherein the nonresistant basil plant comprises sweet basil and the resistant basil plant comprises wild basil.

27. The method of claim 25, wherein the resistant basil plant comprises Ocimum ammericanum.

28. The method of claim 25, wherein the resistant basil plant has one of basil accession numbers PI 500945, PI 500950 and PI 652053.

29. The method of claim 26, wherein the sweet basil plant is Ocimum basilicum.

30. The method of claim 25, wherein the non-resistant basil is selected from the group consisting of O. kilimanadascharicum, O. tenuiflorum, O. basilicum O. basilicum var. anisatum, O. basilicum var. thyrsiflorum, O. basilicum var. citrodorum and O. x citrodorum (Syn O. americanum Lemon Types) O. basilicum var. minimum and hybrids thereof.

31. The method of claim 25, wherein the rescuing comprises:
growing a receptacle separated from a sterile basil plant on MS medium at about 25° C. and then at about 18° C.;
transferring immature seed to MS medium to develop plantlets;
transfer plantlets to rooting medium; and
grow plantlets at 27° C. to obtain fertile basil plants.

32. The method of claim 25, wherein the resistant basil plant is fertile.

33. A basil plant or a seed thereof, produced by the method of claim 25.

34. The basil plant or seed according to claim 33, wherein said introgressed sequence conferring the resistance, is from Ocimum ammericanum.

35. The basil plant or seed according to claim 33, wherein the seed is deposited at NCIMB accession number NCIMB-42946.

36. A basil downy mildew (BDM) resistant cultivated basil plant and/or seed, comprising:
a genomic sequence having one or more introgressed nucleic acid sequences conferring resistance or tolerance to BDM relative to a basil plant of the same species lacking the introgressed nucleic acid sequences.

37. The BDM resistant cultivated basil plant and/or seed of claim 36, wherein said resistant cultivated basil plant is fertile.

38. The BDM resistant cultivated basil plant and/or seed of claim 36, wherein the cultivated basil plant and/or seed is selected from the group consisting of O. kilimanadascharicum, O. tenuiflorum, O. basilicum O. basilicum var. anisatum, O. basilicum var. thyrsiflorum, O. basilicum var. citrodorum and O. x citrodorum (Syn O. americanum Lemon Types) O. basilicum var. minimum and hybrids thereof.

39. The BDM resistant cultivated basil plant and/or seed of claim 36, wherein the cultivated basil plant and/or seed is an Ocimum basilicum plant and/or seed or any hybrid thereof.

40. The BDM resistant cultivated basil plant and/or seed of claim 36, wherein said one or more introgressed sequences are derived from O. americanum.

41. The BDM resistant cultivated basil plant and/or seed of claim 40, wherein the O. americanum is O. americanum var americanum accession number PI 500945.

42. The BDM resistant cultivated basil plant and/or seed of claim 36, wherein said one or more introgressed sequences are located at a distance of less than 30 centrimorgan (cM) from a genetic marker having an amino acid sequence selected from SEQ ID NOs: 1-13.

43. The BDM resistant cultivated basil plant and/or seed of claim 36, wherein said one or more sequences are homozygously introgressed into the BDM resistant cultivated basil plant and/or seed.


SEQUENCE LISTING (13 sequences)

SEQ ID NO: 1 - DNA, 69 bp, Unknown
  SNP marker
  misc_feature (12)..(12): n is “c or g”
tgcaggctacgncttttgaactgctctgtgagaaacgagcatttcatattacagatcggaagagcggtt

SEQ ID NO: 2 - DNA, 66 bp, Unknown
  SNP marker
  misc_feature (9)..(9): n = a or g
  misc_feature (9)..(9): n is a or g
tgcagaagntggaatctagggttttgagcacttctttcgcgagttcgggggaagaaatgacgatta

SEQ ID NO: 3 - DNA, 69 bp, Unknown
  SNP marker
  misc_feature (7)..(7): n is a or g
  misc_feature (36)..(36): n is a or g
tgcagcngtggtgtgagcaggtgacgagagcgagcntagcagcggccggcgaaccagaacagaaatgga

SEQ ID NO: 4 - DNA, 69 bp, Unknown
  SNP marker
  misc_feature (6)..(6): n is c or a
  misc_feature (22)..(22): n is c or t
tgcagnagaagctttagtgcanataatactgatggagatggtttttgcctgacttctgtttgttgtgct

SEQ ID NO: 5 - DNA, 69 bp, Unknown
  SNP marker
  misc_feature (12)..(12): n is t or a
tgcaggacattntgcaaactggaaaaacgattttcatcagctcaactttacagatcggaagagcggttc

SEQ ID NO: 6 - DNA, 66 bp, Unknown
  SNP marker
  misc_feature (53)..(53): n is c or a
  misc_feature (58)..(58): n is c or a
  misc_feature (62)..(62): n is g or a
tgcagaaaacggaatctagggttttcagcacttcttttgctagtttgggtgangaaanaacnatta

SEQ ID NO: 7 - DNA, 69 bp, Unknown
  SNP marker
  misc_feature (24)..(24): n is t or c
tgcagcaaatacggctactgcggnaatggttccgtaggtaaacatatttcccattatcttacagatcgg

SEQ ID NO: 8 - DNA, 69 bp, Unknown
  SNP marker
  misc_feature (34)..(34): n is t or g
  misc_feature (62)..(62): n is t or c
  misc_feature (67)..(67): n is t or a
tgcagcattagtccccgaagctccggatgtgaanatatggtttttctggaaagaaagcgatngaaantc

SEQ ID NO: 9 - DNA, 69 bp, Unknown
  SNP marker
  misc_feature (8)..(8): n is g or a
  misc_feature (47)..(47): n is t or c
tgcagtcnttatatctaatgatgggacaaggactgaaaccagtttcntctgcaaaagcaggtaacatca

SEQ ID NO: 10 - DNA, 69 bp, Unknown
  SNP marker
  misc_feature (24)..(24): n is t or c
tgcagcatggcaccaaacatggtngcgcatataattgcttgcttatttgttatcagcatttgcttctgt

SEQ ID NO: 11 - DNA, 69 bp, Unknown
  SNP marker
  misc_feature (18)..(18): n is a or g
  misc_feature (66)..(66): n is a or t
tgcagcaagagggaggancaaacgacgcttaccgatgaggctgccatgcaagcgctagcgagccanggg

SEQ ID NO: 12 - DNA, 69 bp, Unknown
  SNP marker
  misc_feature (47)..(47): n is g or a
  misc_feature (66)..(66): n is c or g
tgcaggtcgaggagctggtgctcaagagaaagatctacagggtggtntacaagatggatagctctngga

SEQ ID NO: 13 - DNA, 69 bp, Unknown
  SNP marker
  misc_feature (13)..(13): n is a or t
  misc_feature (39)..(39): n is c or t
tgcagttgaatantcattttctttccaaaattgttgagnagttggctgcatagtcaattacagatcgga